(19) United States
(12) Patent Application Publication
(10) Pub. No.: US 2005/0226929 A1
(43) Pub. Date: Oct. 13, 2005

(54) Controlled release opioid analgesic formulation

(76) Inventors: Jianbo Xie, Davie, FL (US); Xiu Xiu Cheng, Weston, FL (US); Chih-Ming Chen, Taipei (TW)

Correspondence Address: HEDMAN & COSTIGAN P.C., 1185 AVENUE OF THE AMERICAS, NEW YORK, NY, 10036, US

(21) Appl. No.: 10/822,627

(22) Filed: Apr. 12, 2004

(51) Int. Cl. A61K 9/22; A61K 9/24; A61K 9/16

(52) U.S. Cl. 424/471

(57) ABSTRACT

A pharmaceutical sustained release formulation for opioid analgesics which can be administered every 12 hours for control of pain in patients suffering from chronic pain.

### BACKGROUND OF THE INVENTION

1. Field of the Invention

The present invention is a novel formulation for the sustained release of an opioid analgesic such as oxycodone, a pharmaceutically acceptable salt, ester or amide thereof. In particular, the present invention relates to a sustained release oral pharmaceutical dosage formulation that can be administered to a patient twice daily or every 12 hours to provide relief from chronic pain.

2. Description of the Related Art

Daily doses of opioid analgesics required to control pain vary greatly from patient to patient and often have to be administered every two or three hours for relief of pain. When multiple daily dosing is required, the problems of proper titration for analgesia become an issue. The continual repeating of multiple daily doses often leaves the patient with unacceptable periods of pain control loss.

Oxycodone hydrochloride is an opioid agonist whose principle therapeutic action is analgesia. Oxycodone hydrochloride and its derivatives are frequently administered to patients for the management of acute and chronic pain where the use of an opioid analgesic is appropriate for more than a few days.

A problem that arises in the administration of oxycodone hydrochloride and its derivatives centers on the drugs inherent short half-life. The elimination half-life of oxycodone in the immediate release form has been reported to be as short as three hours. Subsequently, this leads to multiple daily doses to sustain therapeutic levels of the medication. The required multiple daily dosing leads to the associated inconvenience of frequent administration of the medication and, as with any therapy that requires multiple dosing there is an associated compliance problem. In particular, analgesics are frequently over and under dosed because of the aforementioned reasons.

As related to analgesic therapy, over and under dosing can have dire consequences. The associated problem incurred when administering oxycodone hydrochloride and its derivatives in multiple daily doses may results in poor pain control and/or adverse effects necessitating intervention and increasing health care costs.

Controlled release preparations of oxycodone hydrochloride and its derivatives have been developed to overcome the problems of loss of pain control and to maintain stable plasma concentrations.

The effort to find a formulation which delivers the drug by controlled release which affords a stable plasma level in a sustained release formulation has been well documented.

The following patents describe formulations which include oxycodone hydrochloride and its derivatives in either time or controlled release dosage forms.

Oshlack, et al. U.S. Pat. No. 5,549,912 discloses a controlled release dosage form of oxycodone or a salt thereof in a film coated tablet having a matrix core made with a combination of natural or synthetic polymers which is incorporated herein by reference. Leslie U.S. Pat. No. 4,235,870 describes a slow release pharmaceutical composition prepared with a sustained release matrix comprising hydroxyalkyl cellulose components and higher aliphatic alcohols. Other opioid analgesic controlled release formulations are disclosed in U.S. Pat. Nos.: 4,443,428; 4,861,598; 4,970,075; 5,266,331; 5,478,577 and 5,672,360.

However, the need for a novel sustained release formulation of oxycodone hydrochloride which will effectively maintain plasma concentrations of the drug at more constant levels still remains.

### SUMMARY OF THE INVENTION

The present invention provides, in its principle embodiment, a novel sustained release tablet dosage formulation substantially improving the efficiency and quality of pain management.

It is another object of the present invention to provide an opioid analgesic formulation which substantially improves the efficiency and quality of pain management.

It is another object of the present invention to provide a novel method and formulation which substantially reduces the daily dosages required to control pain in substantially all patients requiring relief from chronic pain.

It is yet another object of the present invention to provide a method for substantially reducing the time and resources needed to titrate patients requiring pain relief on opioid analgesics.

These and other objectives are met by the present invention which is a sustained release oral pharmaceutical tablet comprising:
(1) a core comprising:
   (a) an opioid analgesic;
   (b) at least one pharmaceutical excipient; and
(2) a delayed release coating comprising:
   (a) a first enteric coating agent;
   (b) a second enteric coating agent;
   (c) optionally a plasticizer;
   (d) optionally an inert processing aid; and
(3) an immediate release drug layer comprising:
   (a) an opioid analgesic;
   (b) a binder; and
(4) optionally a cosmetic coating.

The pharmaceutical excipients that can be used in the core of the present invention include, but are not limited to binders, diluents, lubricants, glidants, emulsifiers, osmopolymers, osmotic agents, glidants, flavoring agents and combinations of the foregoing. A more complete list of common pharmaceutical excipients can be found in The United States Pharmacopeia XV, Remington, The Science and Practice of Pharmacy, 20^{th} ed. and the Handbook of Pharmaceutical Excipients, 3^{rd} ed., all of which are incorporated herein by reference.

In a preferred embodiment of the present invention the core comprises the opioid analgesics and a combination of a binder, a diluent, a lubricant and a glidant.

### DETAILED DESCRIPTION

Opioid analgesics, as used in this application, refer to compounds such as, alfentanil, allyprodine, alphaprodine, anileridine, benzylmorphine, bezitramide, buprenorphine, btorphanol, clonitazene, codeine, cyclazocine, desomorphine, dextromoramide, dezocine, diampromide, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narcine, nicomorphine, norlevorphanol, normethadone, nalorphine, normorphine, norpipanone, opium, oxycodone, oxymorphone, papaveretum, pentazocine, phenadoxone, phenmorphan, phenazocine, phenoperidine, piminodine, piritramide, propheptazine, promedol, properidine, propiram, propoxyphene, sufentanil, tramadol, tilidine and salts thereof. The preferred opioid analgesics are buprenorphine, codeine, dihydrocodeine, dihydromorphine, hydromorphone, morphine, oxycodone and salts of the foregoing.

The amount of opioid analgesic employed in the dosage form of the present invention, can be easily determine once the opioid analgesic is selected based upon the published literature and history of the drug. For example, typical therapeutic amounts of hydromorphone range from about 4 mg to about 64 mg of the hydrochloride salt, typical therapeutic amounts of morphine range from about 5 mg to about 800 mg and typical therapeutic amount of oxyocdone range from about 5 mg to abut 400 mg for the hydrochloride salt.

The opioid analgesic may comprise about 1% to about 50% of the total weight of the core and preferably about 5 to about 40% of the total weight of the core.

If the core employs a binding agent, it can be any type of material commonly known in the pharmaceutical arts. The binding agent can be polymeric, non-polymer, water soluble or water insoluble. Preferably, the binding agent employed in the core is a water soluble, and most preferably gels or swells in the presence of water. Some of the commonly known binders are acacia, alginic acid, sodium carboxymethylcellulose, microcrystalline cellulose, dextrin, ethylcellulose, gelatin, liquid glucose, polyvinyl pyrrolidone, carboxyvinyl polymer, methylcellulose, hydroxymethyl cellulose, low molecular weight polyethylene oxide polymers, hydroxyethyl cellulose, hydroxypropyl cellulose, hydroxypropyl methylcellulose (HPMC), gums, acrylate polymers, methacrylate polymers, maltodextrin and mixtures of the foregoing. The preferred binding agent for use in the core should be water soluble and have a viscosity of greater than 50,000 mPa, preferably greater than 75,000 mPa when tested in a 2% aqueous solution at 20° C. An example of a typical binder that is water soluble and has a viscosity of greater than 75,000 mPa is the hydroxypropyl methylcellulose product sold by Dow Chemical under the tradename METHOCEL® K-100M. Other binders that can be used in the present invention are the osmopolymers described in U.S. Pat. Nos. 5,082,668; 4,783,337; 4,612,0008 and 4,327,725 which are incorporated herein by reference.

The amount of binder in the core may comprise 0.5 to about 50% of the total weight of the core and preferably about 1 to about 40% of the total weight of the core.

The core may optionally contain a diluent or filler. If a diluent or filler is employed in the core, it can be any type of diluent commonly known in the art such as sugars, starches or vegetable oils. Examples of some preferred diluent are lactose monohydrate, calcium carbonate, calcium sulfate, microcrystalline cellulose, calcium phosphate, dextrin, dextrose, maltitol, maltose, starch, sucrose or talc. In a preferred embodiment of the present invention, the diluent used in the core of the tablet is lactose monohydrate. If a diluent is used in the core, the amount should be about 1 to about 95% based on the total weight of the core, preferably 25 to 90% based on the total weight of the core, and most preferably about 50 to about 90% based on the total weight of the core.

The core may also optionally contain a lubricant. Lubricants are used to facilitate tablet manufacturing of the formulation. Some examples of suitable lubricants include, talc, glyceryl monostearates, calcium stearate, magnesium stearate, stearic acid, glyceryl behenate, and polyethylene glycol, and are preferably present at no more than approximately about 0.05 to about 15% based upon the total weight of the core, preferably about 0.1 to about 10% based upon the total weight of the core and most preferably about 0.5 to about 5% based upon the total weight of the core.

The core may also comprise a flow aid or glidant. A glidant is an excipient that improves the flow characteristics of a compressible powder such as tablet ingredients or granules. Two of the most common glidants are colloidal silicon dioxide (CAB-O-SIL®) and Quso (also known as Phila Quartz). The amount of glidant that can be used in the present invention ranges from about 0.1 to about 5 weight percent.

The core of the present invention is preferably formed by mixing the core ingredients and tableting the mixture using techniques commonly known in the art. The core may also be formed by granulating the core ingredients and compressing the granules into a tablet. The tableting can be preformed on any type of tableting apparatus such as a rotary press. The tablet core can optionally be coated with a seal coat prior to application of the delayed release coating.

In a preferred embodiment the core is subsequently coated with a delayed release coating. This delayed release coating is applied by conventional coating techniques, such as pan coating or fluid bed coating using solution, suspension or dispersion of polymeric material in water or suitable organic solvents. A particularly preferred delayed release coating employs pH dependent materials. The term “pH dependent” as used in this application refers to materials that do not dissolve or degrade in the acidic stomach environment. Preferably a pH dependent agent is selected so that when it is incorporated into the delayed release coating it facilitates or delays the release of the pharmaceutically active ingredient from the core into environments with a pH of 5 or greater, more preferably at pH 6 or greater and most preferably at pH 7 of greater. The delayed release coating comprises about 2 to about 50 weight percent of the coated core, preferably about 5 to about 30 weight percent of the coated core. Preferably, the pH dependent material employed in the present invention is selected from the group consisting of zein, methacrylic acid copolymers, cellulose acetate phthalate, hydroxypropyl methylcellulose phthalate, hydroxypropyl methylcellulose acetate succinate, cellulose acetate trimellitate, polyvinyl acetate phthalate or mixtures thereof. The amount of pH dependent material in the delayed release coating is preferably about 30 to about 80 weight percent, preferably about 35 to about 60 weight percent based on the total weight of the delayed release coating.

In a preferred embodiment of the present invention, a mixture of two different pH dependent materials is employed such as a mixture of zein and a methacrylic acid copolymer. When the two pH dependent materials are employed they should be selected so that they begin dissolving at different pH points. For example, one of the pH dependent materials should begin dissolving around a pH of about 5 to about 6, or a pH of about 6 to about 7 while the second pH dependent material should begin to degrade in the gastrointestinal tract or begin to dissolve around a pH above 7, preferably above 8, and more preferably above 9 and most preferably between a pH of about 11 to about 12. This mixture helps to control the hydration of the core and thereby the release of the drug from the core.

As mentioned above, one embodiment of the present invention employs a combination of zein and a methacrylic acid copolymer. The methacrylic acid copolymer is selected from the group of pH dependent coating polymers, preferably Eudragit S, and most preferably Eudragit S 100. These methacrylic acid copolymers begin dissolving in the range of about pH 6 to about pH 7, while zein begins to dissolve around pH 11.5. The preferred ratio of zein to methacrylic acid copolymer is 1:5 to 5:1 with the most preferred ratio being 2:1 to 4:1. Varying the ratio of zein to methacrylic acid copolymer will also aid in controlling the hydration rate of the core and thereby the drug release.

The delayed release coating may also preferably contains plasticizers. Plasticizers which may be used include any of those known to those skilled in the art, including but not limited to, acetyltributyl citrate, triacetin, acetylated monoglyceride, rape oil, olive oil, sesame oil, acetyltriethyl citrate, glycerin sorbitol, diethyloxalate, diethylmalate, diethylfumerate, dibutylsuccinate, diethylmalonate, dioctylphthalate, dibutylphthalate, dibutylsebacate, triethyl citrate, tributylcitrate, glyceroltributyrate, polyethylene glycol, propylene glycol and mixtures thereof. The preferred plasticizer is acetyltributyl citrate. The amount of plasticizer employed in the controlled release coating can range from about 0.1 to about 15% based on the total weight of the controlled release coating.

The delayed release coating may also include an inert processing aid or an anti-sticking agent such as those selected from the group consisting of talc, colloidal silica dioxide, magnesium stearate, magnesium silicate, glyceryl monostearates, calcium stearate or steric acid. The preferred inert processing aid or anti-sticking agent is talc. If an anti-sticking agent is employed in the controlled release coating, the amount employed should range from about 20 to about 70 percent, and most preferably 30-60% based on the total weight of the coating.

In a preferred embodiment of the present invention, a second coating is applied to the controlled release coating to form an immediate release portion of the dosage formulation. This second or immediate release layer is comprised of the opioid analagesic and a binder that preferably is a pharmaceutically acceptable water-soluble or rapidly dispersing material. The binder employed can be any type of binding agent commonly known in the art such as those described above. In a preferred embodiment of the present invention, the binder for the immediate release coating is a water soluble polymer such as hydroxypropyl methylcellulose, commercially available from Dow Chemicals under the trade name METHOCEL® E-5. Other binders for the immediate release layer are commercially available from Colorcon under the tradename OPADRY®, such as OPADRY® clear, OPADRY® white or OPADRY® yellow. The binder in the immediate release layer should comprise about 5% to about 70%, preferably about 10% to about 60% based upon the total weight of the immediate release layer. In addition to the opioid analgesic and binder, the immediate release layer may contain other conventional pharmaceutical additives such as coloring agents, dyes, plasticizers, surfactants, emulsifiers, stabilizers or mixtures of the foregoing.

Finally, the immediate release coating may be coated with a seal coat, polishing coat or color coat using any of the methods commonly known in the industry. The dissolution profile of the dosage form prepared according to the present invention with an immediate release coating should exhibit the following dissolution profile when tested in a USP type 2 (paddle) apparatus at 75 rpms in 900 ml of water with a of pH 7.5 and at 37° C.

DRUG RELEASED

| Time (hours) | Preferred | Most Preferred |
|---|---|---|
| 1 | 15-70% | 20-65% |
| 3 | NLT 25% | NLT 35% |
| 6 | NLT 50% | NLT 60% |
| 8 | NLT 70% | NLT 80% |

*NLT = Not Less Than

### DESCRIPTION OF A PREFERRED ENBODIMENT

The present invention will be further illustrated by the following examples.

### EXAMPLE 1

A controlled release tablet containing approximately 80 mg of oxycodone HCl and in accordance with the present invention is prepared as follows:

(a) Core

0.2807 kg of oxycodone HCl, 0.050 kg of hydroxypropyl methylcellulose 2208 USP (METHOCEL® K100 M Premium), 1.643 kg of lactose monohydrate and 0.020 kg of colloidal silicon dioxide (CAB-O-SIL®, M-5) are delumped by passing the compounds through a 20 mesh screen and adding them to a twin shell blender. The materials are blended for approximately 20 minutes at 23 rpms. 0.020 kg of magnesium stearate is then delumped by passing it through a 30 mesh screen and is added to the twin shell blender. The ingredients are then blended for an additional 5 minutes at a speed of about 23 rpms. Once the ingredients are blended, they are pressed into tablets using a Betapress, equipped with a 11/32″ round standard concave die. The target weight of the tablet core is about 300 mg with a hardness of about 10±3 kp.

(b) Delayed Release Coating

A delayed release coating suspension is prepared by mixing 4.879 kg of isopropyl alcohol USP and 0.921 kg of purified water, USP in a 5 gallon container. Using a mechanical stirrer, 0.133 kg of methacrylic acid copolymer (EUDRAGIT® S100) and 0.399 kg of zein are slowly added to the isopropyl alcohol and water solution and stirred until a clear solution is obtained. Once the clear solution is obtained, 0.044 kg of acetyltributyl citrate USP is added to the solution. An additional 0.544 kg of isopropyl alcohol is used to rinse the container used to hold the acetyltributyl citrate. The isopropyl alcohol rinse is added to the delayed release coating solution and stirred for an additional 2 to 5 minutes. 0.532 kg of talc is added to the delayed release coating solution and mixed with the mechanical stirred for about 5 minutes or until the talc is completely dispersed. The resulting delayed release coating suspension is stirrer continuously until it is consumed in the coating process.

1.6 kg of the tablet cores prepared in step (a) and 8.376 kg of similar sized and shaped placebo core are placed into an Ohara Lab Coat II pan coater equipped with a 30″ pan and coated with the delayed release coating suspension using the following conditions:

| | |
|---|---|
| Exhaust temperature: | 30 ± 5° C. |
| Pan speed: | 8-10 rpms |
| Spray Rate: | 30-40 ml/min |

Once the delayed release coating suspension has been consumed, the delayed release coated cores are place on drying trays and dried for about 16 hours at 60±5° C.

(c) Immediate Release Coat

An immediate release drug coating solution is prepared by adding 1.361 kg of hydroxypropyl methylcellulose (METHOCEL® E5 PREMIUM) to 27.22 kg of purified water while stirring with a mechanical stirrer. 1.361 kg of oxycodone HCl is added to the hydroxypropyl methylcellulose solution while stirring with a mechanical stirrer. The resulting immediate release drug coating solution is continuously stirred until the coating solution is consumed.

1.688 kg of the oxycodone HCl enteric coated tablets from step (b) above and 8.0 kg of placebo tablets are placed in an Ohara pan coater equipped with a 24″ pan and two spray guns. The tablets are coated with the immediate release drug coating solution using the following conditions:

| | |
|---|---|
| Exhaust temperature: | 40 ± 1° C. |
| Pan speed: | 11 rpms |
| Spray Rate: | 25-30 ml/min |

Once the immediate release drug coating solution is consumed, the coated tablets are allowed to dry in the pan coater for about 10 minutes.

The dried immediate release coated tablets are finally color coated with an aqueous solution of OPADRY® Orange (YS-1-13664A).

The resulting tablets were tested using a USP apparatus 2 with 900 ml of pH 7.5 potassium phosphate buffer at 75 rpms. The dissolution test results were:

| TIME | PERCENT DISSOLVED |
|---|---|
| 1.0 | 51 |
| 2.0 | 60 |
| 3.0 | 84 |
| 4.0 | 98 |
| 6.0 | 108 |
| 8.0 | 109 |
| 12.0 | 109 |

The tablets produced in this example were also tested in six human subjects along with OXYCOTIN® brand oxycodone HCl tablets. The results for the Fasting study showed a Cₘₐₓ of 1.15 and a AUC of 1.04. The results for the Fed study showed a Cₘₐₓ of 1.058 and a AUC of 1.065.

### EXAMPLE 2

A controlled release tablet containing approximately 80 mg of oxycodone HCl and in accordance with the present invention is prepared as follows:

(a) Core

0.2807 kg of oxycodone HCl, 0.10 kg of hydroxypropyl methylcellulose 2208 USP (METHOCEL® K100 M Premium), 1.593 kg of lactose monohydrate and 0.020 kg of colloidal silicon dioxide (CAB-O-SIL®, M-5) are delumped by passing the compounds through a 20 mesh screen and adding them to a twin shell blender. The materials are blended for approximately 20 minutes at 23 rpms. 0.020 kg of magnesium stearate is then delumped by passing it through a 30 mesh screen and is added to the twin shell blender. The ingredients are then blended for an additional 5 minutes at a speed of about 23 rpms. Once the ingredients are blended, they are pressed into tablets using a Betapress, equipped with a 11/32″ round standard concave die. The target weight of the tablet core is about 300 mg with a hardness of about 10±3 kp.

(b) Delayed Release Coating

A delayed release coating suspension is prepared by mixing 4.879 kg of isopropyl alcohol USP and 0.921 kg of purified water, USP in a 5 gallon container. Using a mechanical stirrer, 0.133 kg of methacrylic acid copolymer (EUDRAGIT® S 100) and 0.399 kg of zein are slowly added to the isopropyl alcohol and water solution and stirred until a clear solution is obtained. Once the clear solution is obtained, 0.044 kg of acetyltributyl citrate USP is added to the solution. An additional 0.544 kg of isopropyl alcohol is used to rinse the container used to hold the acetyltributyl citrate. The isopropyl alcohol rinse is added to the delayed release coating solution and stirred for an additional 2 to 5 minutes. 0.532 kg of talc is added to the delayed release coating solution and mixed with the mechanical stirrer for about 5 minutes or until the talc is completely dispersed. The resulting delayed release coating suspension is stirred continuously until it is consumed in the coating process.

1.8 kg of the tablet cores prepared in step (a) and 8.176 kg of similar sized and shaped placebo cores are placed into an Ohara Lab Coat II pan coater equipped with a 24″ pan and coated with the delayed release coating suspension using the following conditions:

| | |
|---|---|
| Exhaust temperature: | 30 ± 5° C. |
| Pan speed: | 8-10 rpms |
| Spray Rate: | 30-40 ml/min |

Once the delayed release coating suspension has been consumed, the enteric coated cores are place on drying trays and dried for about 16 hours at 60±5° C.

(c) Immediate Release Coat

An immediate release drug coating solution is prepared by adding 1.361 kg of hydroxypropyl methylcellulose (METHOCEL® E5 PREMIUM) to 27.22 kg of purified water while stirring with a mechanical stirrer. 1.361 kg of oxycodone HCl is added to the hydroxypropyl methylcellulose solution while stirring with a mechanical stirrer. The resulting immediate release drug coating solution is continuously stirred until the coating solution is consumed.

1.688 kg of the oxycodone HCl enteric coated tablets from step (b) above and 8.0 kg of placebo tablets are placed in an Ohara pan coater equipped with a 24″ pan and two spray guns. The tablets are coated with the immediate release drug coating solution using the following conditions:

| | |
|---|---|
| Exhaust temperature: | 40 ± 2° C. |
| Pan speed: | 11 rpms |
| Spray Rate: | 30 ml/min |

Once the immediate release drug coating solution is consumed, the coated tablets are allowed to dry in the pan coater for about 10 minutes.

The dried immediate release coated tablets are finally color coated with an aqueous solution of OPADRY® Orange (YS-1-13664A).

The resulting tablets were tested using a USP apparatus 2 with 900 ml of pH 7.5 potassium phosphate buffer at 75 rpms. The dissolution test results were:

| TIME | PERCENT DISSOLVED |
|---|---|
| 1.0 | 49 |
| 2.0 | 58 |
| 3.0 | 69 |
| 4.0 | 78 |
| 6.0 | 99 |
| 8.0 | 109 |
| 12.0 | 109 |

The tablets produced in this example were also tested in six human subjects along with OXYCOTIN® brand oxycodone HCl tablets. The results for the Fasting study showed a Cₘₐₓ of 1.2032 and a AUC of 1.068. The results for the Fed study showed a Cₘₐₓ of 1.073 and a AUC of 1.1

While certain preferred and alternative embodiments of the invention have been set forth for purposes of disclosing the invention, modifications to the disclosed embodiments may occur to those who are skilled in the art. Accordingly, the appended claims are intended to cover all embodiments of the invention and modifications thereof which do not depart from the spirit and scope of the invention.

1. A sustained release oral pharmaceutical dosage formulation comprising:
(a) a core comprising:
(i) an opioid analgesic;
(ii) at least one pharmaceutical excipient; and
(b) a delayed release coating surrounding the core comprising:
(i) a first enteric coating agent;
(ii) a second enteric coating agent;
(iii) optionally a plasticizer;
(iv) optionally an inert processing aid; and
(c) an immediate release drug layer comprising:
(i) an opioid analgesic;
(ii) a binder; and
(d) optionally a cosmetic coating.

2. The sustained release dosage formulation as defined in claim 1 wherein the opioid analgesic is selected from the group consisting of buprenorphine, codeine, dihydrocodeine, dihydromorphine, hydromorphone, morphine, oxycodone and salts of the foregoing.

3. The sustained release dosage formulation as defined in claim 2 wherein the opioid analgesic is oxycodone or a pharmaceutically acceptable salt thereof.

4. The sustained release dosage formulation as defined in claim 1 wherein the pharmaceutical excipient in the core is selected from the group consisting of binders, diluents, lubricants, emulsifiers, osmopolymers, osmotic agents, glidants, flavoring agents and combinations of the foregoing.

5. The sustained release dosage formulation as defined in claim 1 wherein the pharmaceutical excipient in the core comprises a binder and a diluent.

6. The sustained release dosage formulation as defined in claim 5 wherein the pharmaceutical excipient in the core further comprises a glidant and a lubricant.

7. The sustained release dosage formulation as defined in claim 5 wherein the binder is an osompolymer.

8. The sustained release dosage formulation as defined in claim 5 wherein the binder is water soluble and has a viscosity of greater than 50,000 mPa when tested in a 2% aqueous solution at 20° C.

9. The sustained release dosage formulation as defined in claim 5 wherein the binder is water soluble and has a viscosity of greater than 75,000 mPa when tested in a 2% aqueous solution at 20° C.

10. The sustained release dosage formulation as defined in claim 1 wherein the first enteric coating agent begins to dissolve at a pH of about 5 to about 6 and the second enteric agent begins to dissolve at a pH of above 7 or is degraded in the gastrointestinal tract.

11. The sustained release dosage formulation as defined in claim 1 wherein the first enteric coating agent begins to dissolve at a pH of about 6 to about 7 and the second enteric agent begins to dissolve at a pH of above 8 or is degraded in the gastrointestinal tract.

12. The sustained release dosage formulation as defined in claim 10 wherein the second enteric agent begins to dissolve at a pH of about 11 to about a pH of 12.

13. The sustained release dosage formulation as defined in claim 10 wherein the ratio of first enteric agent to the second enteric agent is about 1:5 to 5:1.

14. The sustained release dosage formulation as defined in claim 13 wherein the ratio of first enteric agent to the second enteric agent is about 1:2 to about 1:4.

15. The sustained release dosage formulation as defined in claim 1 wherein the inert processing aid comprises about 20 to about 70 percent of the total weight of the delayed release coating.

16. The sustained release dosage formulation as defined in claim 15 wherein the inert processing aid comprises about 30 to about 60 percent of the total weight of the delayed release coating.

17. A sustained release oral pharmaceutical dosage formulation comprising:
(a) a core comprising:
(i) an opioid analgesic;
(ii) a diluent;
(iii) a binder that is water soluble and has a viscosity of greater than 50,000 mPa when tested in a 2% aqueous solution at 20° C.; and
(b) a delayed release coating surrounding the core comprising:
(i) a first enteric coating agent that begins to dissolve at a pH of about 5 to about 6;
(ii) a second enteric coating agent that begins to dissolve at a pH of above 8;
(iii) an inert processing aid;
(iv) optionally a plasticizer; and
(c) an immediate release drug layer comprising:
(i) an opioid analgesic;
(ii) a binder; and
(d) optionally a cosmetic coating.

18. The sustained release dosage formulation as defined in claim 17 wherein the opioid analgesic is selected from the group consisting of buprenorphine, codeine, dihydrocodeine, dihydromorphine, hydromorphone, morphine, oxycodone and salts of the foregoing.

19. The sustained release dosage formulation as defined in claim 18 wherein the opioid analgesic is oxycodone or a pharmaceutically acceptable salt thereof.

20. The sustained release dosage formulation as defined in claim 17 wherein the binder in the core has a viscosity of greater than 75,000 mPa when tested in a 2% aqueous solution at 20° C.

21. The sustained release dosage formulation as defined in claim 17 wherein the first enteric coating agent begins to dissolve at a pH of about 6 to about 7 and the second enteric agent begins to dissolve at a pH of above 9.

22. The sustained release dosage formulation as defined in claim 17 wherein the second enteric agent begins to dissolve at a pH of about 11 to about a pH of 12.

23. The sustained release dosage formulation as defined in claim 17 wherein the ratio of first enteric agent to the second enteric agent is about 1:5 to 5:1.

24. The sustained release dosage formulation as defined in claim 17 wherein the ratio of first enteric agent to the second enteric agent is about 1:2 to about 1:4.

25. The sustained release dosage formulation as defined in claim 17 wherein the inert processing aid comprises about 20 to about 70 percent of the total weight of the delayed release coating.

26. The sustained release dosage formulation as defined in claim 25 wherein the inert processing aid comprises about 30 to about 60 percent of the total weight of the delayed release coating.

27. A sustained release oral pharmaceutical dosage formulation consisting essentially of:
(a) a core comprising:
(i) oxycodone or a pharmaceutically acceptable salt;
(ii) a diluent;
(iii) a binder that is water soluble and has a viscosity of greater than 50,000 mPa when tested in a 2% aqueous solution at 20° C.;
(iv) a lubricant;
(v) a glidant; and
(b) a delayed release coating surrounding the core consisting essentially of:
(i) a first enteric coating agent that begins to dissolve at a pH of about 5 to about 6 or is degraded in the gastrointestinal tract;
(ii) a second enteric coating agent that begins to dissolve at a pH of above 7 or is degraded in the gastrointestinal tract;
(iii) about 20 to about 70 percent of the total weight of the delayed release coating of an inert processing aid;
(iv) optionally a plasticizer; and
(c) an immediate release drug layer consisting essentially of:
(i) oxycodone or a pharmaceutically acceptable salt;
(ii) a binder; and
(d) optionally a cosmetic coating.

28. The sustained release dosage formulation as defined in claim 27 wherein the binder in the core has a viscosity of greater than 75,000 mPa when tested in a 2% aqueous solution at 200 C.

29. The sustained release dosage formulation as defined in claim 27 wherein the first enteric coating agent begins to dissolve at a pH of about 6 to about 7 and the second enteric agent begins to dissolve at a pH of above 8 or is degraded in the gastrointestinal tract.

30. The sustained release dosage formulation as defined in claim 27 wherein the second enteric agent begins to dissolve at a pH of about 11 to about a pH of 12.

31. The sustained release dosage formulation as defined in claim 27 wherein the ratio of first enteric agent to the second enteric agent is about 1:5 to 5:1.

32. The sustained release dosage formulation as defined in claim 27 wherein the ratio of first enteric agent to the second enteric agent is about 1:2 to about 1:4.

33. The sustained release dosage formulation as defined in claim 27 wherein the inert processing aid comprises about 30 to about 60 percent of the total weight of the delayed release coating.
